(19) United States
(12) Patent Application Publication
(10) Pub. No.: US 2018/0358127 A1
(43) Pub. Date: Dec. 13, 2018

(54) METHOD FOR PREDICTING AUTISM

(71) Applicant: RENSSELAER POLYTECHNIC INSTITUTE, Troy, NY (US)

(72) Inventors: Daniel Howsmon, Troy, NY (US); Uwe Kruger, Ballston Lake, NY (US); Juergen Hahn, Ballston Lake, NY (US)

(73) Assignee: RENSSELAER POLYTECHNIC INSTITUTE, Troy, NY (US)

(21) Appl. No.: 16/002,329

(22) Filed: Jun. 7, 2018

Related U.S. Application Data
(60) Provisional application No. 62/516,288, filed on Jun. 7, 2017.

(51) Int. Cl. G16H 50/30

(52) U.S. Cl. CPC G16H 50/30

(57) ABSTRACT

Methods and systems for detecting an autism state are disclosed. A plurality of data arrays are received, each including a plurality of values. Each of the plurality of values represent a concentration of a different metabolite. A score for each of the plurality of data arrays is calculated based on a relationship between the plurality of values of each of the respective plurality of data arrays. The score for each of the plurality of data arrays is classified into an autism class and a neurotypical class. A test score for a test data array is calculated based on a relationship between the plurality of test values and can then be grouped into one of the autism class and the neurotypical class. The system thus can use biomarkers identified in a metabolic pathway, such as abnormalities in folate-dependent one-carbon metabolism (FOCM) and transsulfuration (TS), to identify patients with a high likelihood of having autism.

### CROSS REFERENCE TO RELATED APPLICATION(S)

This application claims the benefit of U.S. Provisional Application No. 62/516,288, filed Jun. 7, 2017, which is incorporated by reference as if disclosed herein in its entirety.

### BACKGROUND OF THE DISCLOSURE

Autism Spectrum Disorder (ASD) can encompass a large group of early-onset neurological diseases that can include difficulties with social communication, interaction, and expression of restricted repetitive behaviors and interests. In addition to these defining behavioral symptoms, individuals with ASD can frequently have one or more co-occurring conditions. The conditions can include intellectual disability, ADHD, speech and language delays, psychiatric diagnoses, epilepsy, sleep disorders, and gastrointestinal problems. ASD is estimated to affect about 1.7% of the population and disproportionately affects males. ASD is associated with an impaired quality of life. The lifetime cost of supporting an individual with ASD amounts to about $1.4-2.4 MM, depending on co-existing disorders.

ASD can have a strong genetic component, but environmental effects have also recently emerged as important contributors to the etiology and pathophysiology of ASD in at least a subpopulation of cases. Early twin studies suggested that the heritability of ASD was 80-90%; however, twin studies since 2010 suggest a lower heritability of only 37-55%. Despite this high genetic association, only about 15% of ASD cases have a known genetic source.

No generally accepted biomarkers for the diagnosis or diagnosis of the severity of ASD exist to date. Instead, diagnostic evaluation involves a multi-disciplinary team of doctors usually including a pediatrician, psychologist, speech and language pathologist, and occupational therapist.

### SUMMARY OF THE DISCLOSURE

According to at least one aspect of the disclosure, a system to determine an autism state can include a data processing system that can execute a classifier and a scoring engine. The data processing system can receive a plurality of data arrays. Each of the data arrays can include a plurality of values. Each of the plurality of values can represent a concentration of a different metabolite. The data processing system can calculate a score for each of the plurality of data arrays based on a relationship between the plurality of values of each of the respective plurality of data arrays. The data processing system can classify the score for each of the plurality of data arrays into an autism class and a neurotypical class. The data processing system can receive a test data array that can include a plurality of test values. Each of the plurality of test values can represent the concentration of the different metabolites. The data processing system can calculate a test score for the test data array based on a relationship between the plurality of test values. The data processing system can group the test score into one of the autism class and the neurotypical class based on the test score for the test data array.

In some implementations, the plurality of values can represent the concentration of one of Methionine, SAM, SAH, SAM/SAH, 8-OHG, Adenosine, Homocysteine, Cysteine, γ-L-Glutamyl-L-cysteine (Glu.-Cys.), L-Cysteine-L-Glycine (Cys.-Gly.), tGSH, fGSH, GSSG, fGSH/GSSG, tGSH/GSSG, Chlorotyrosine, Nitrotyrosine, Tyrosine, Tryptophane, fCystine, fCysteine, fCystine/fCysteine, a percent of DNA methylation, or a percent of oxidized glutathione, or combinations thereof. In other implementations, the test data array includes a concentration value of each of DNA methylation, 8-OHG, γ-L-Glutamyl-L-cysteine (Glu.-Cys.), fCystine/fCysteine, Chlorotyrosine, and tGSH/GSSG, and the percent of oxidized glutathione.

In some implementations, the data processing system is further configured to calculate the score for each of the plurality of data arrays using Fisher Discriminant Analysis or similar machine learning techniques used for classification such as support vector machines, PCA, regression trees, etc. The data processing system can define a boarder threshold between the autism class and the neurotypical class. The data processing system can group, responsive to the test score being below the boarder threshold, the test score into the autism class. The boarder threshold can be nonlinear.

In some implementations, the data processing system can be configured to determine a weight for each of the plurality of values. The data processing system can calculate the score for each of the plurality of data arrays using the weight for each of the plurality of values.

According to at least one aspect of the disclosure, a method to determine an autism state can include receiving a plurality of data arrays. Each of the data arrays can include a plurality of values. Each of the plurality of values can represent a concentration of a different metabolite. The method can include calculating a score for each of the plurality of data arrays based on a relationship between the plurality of values of each of the respective plurality of data arrays. The method can include classifying the score for each of the plurality of data arrays into an autism class and a neurotypical class. The method can include receiving a test data array that can include a plurality of test values. Each of the plurality of test values can represent the concentration of the different metabolites. The method can include calculating a test score for the test data array based on a relationship between the plurality of test values. The method can include grouping the test score into one of the autism class and the neurotypical class based on the test score for the test data array.

In some implementations, the plurality of values can represent the concentration of one of Methionine, SAM, SAH, SAM/SAH, 8-OHG, Adenosine, Homocysteine, Cysteine, γ-L-Glutamyl-L-cysteine (Glu.-Cys.), L-Cysteine-L-Glycine (Cys.-Gly.), tGSH, fGSH, GSSG, fGSH/GSSG, tGSH/GSSG, Chlorotyrosine, Nitrotyrosine, Tyrosine, Tryptophane, fCystine, fCysteine, fCystine/fCysteine, a percent of DNA methylation, or a percent of oxidized glutathione. In other implementations, the test data array includes a concentration value of each of DNA methylation, 8-OHG, γ-L-Glutamyl-L-cysteine (Glu.-Cys.), fCystine/fCysteine, Chlorotyrosine, and tGSH/GSSG, and the percent of oxidized glutathione. In some implementations, the test data array can include a concentration value of each of SAM, SAH, SAM/SAH, Adenosine, Homocysteine, Glu-Cys, tGSH/GSSG, a percent of oxidized glutathione.

The method can include calculating the score for each of the plurality of data arrays using Fisher Discriminant Analysis. The method can include defining a boarder threshold between the autism class and the neurotypical class. The method can include grouping, responsive to the test score being below the border threshold, the test score into the autism class. The boarder threshold can be nonlinear. The method can include determining a weight for each of the plurality of values. The method can include calculating the score for each of the plurality of data arrays using the weight for each of the plurality of values.

### BRIEF DESCRIPTION OF THE DRAWINGS

The figures, described herein, are for illustration purposes only. In the drawings, like reference characters generally refer to like features, functionally similar and/or structurally similar elements throughout the various drawings. The drawings are not necessarily to scale, emphasis instead being placed upon illustrating the principles of the teachings. The drawings are not intended to limit the scope of the present teachings in any way. The system and method may be better understood from the following illustrative description with reference to the following drawings in which:

FIG. 1 illustrates a block diagram of an example system to determine an autism state.

FIG. 2 illustrates a block diagram of an example method for diagnosing autism using the example system illustrated in FIG. 1.

FIG. 3 illustrates a plot of the scores for each of the patients' data arrays and the estimated PDF for each of the classes.

FIG. 4 illustrates a plot of the probability distribution functions of the autism, neurotypical, and sibling classes.

FIG. 5 illustrates a bar plot of the maximum C-statistic for all combinations of a given number of metabolite concentration values.

FIG. 6A illustrates a plot of the scores for each of the patients' data arrays and the estimated probability distribution function for each of the classes using data arrays with a reduced number of concentration values.

FIG. 6B illustrates the cross-validated confusion matrix for the separation of the autism and neurotypical classes.

FIG. 7A illustrates a bar graph of the maximum cross-validated R² for a number of variables.

FIG. 7B illustrates a scatter pot of the cross-validated model predictions versus actual data points for the combination of five variables.

### DETAILED DESCRIPTION

The various concepts introduced above and discussed in greater detail below can be implemented in any of numerous ways, as the described concepts are not limited to any particular manner of implementation. Examples of specific implementations and applications are provided primarily for illustrative purposes.

The system described herein can use biomarkers identified in the metabolic pathway to identify patients with a high likelihood of having autism. For example, abnormalities in at least one of folate-dependent one-carbon metabolism (FOCM) and transsulfuration (TS) can reflect predisposition to ASD. FOCM contributes to epigenetic gene expression through DNA methylation and TS is the major contributor to intracellular redox status.

Mutations or altered expression levels of several genes in these pathways can be associated with an increased risk of ASD. Adenylosuccinate lyase (ADSL) deficiency can lead to a purely genetic form of autism by re-directing a large proportion of FOCM toward purine synthesis to compensate for a reduction in de novo purine synthesis. Methylenetetrahydrofolate reductase (MTHFR) can be responsible for generating 5-methyltetrahydrofolate, which in turn can be responsible for re-methylating homocysteine to methionine. In particular, the C677T polymorphism can increase ASD liability, especially in countries where prenatal folate supplementation is low. Mutations in folate carrier (RFC1), transcobalamin II (TCII), serine hydroxymethyltransferase I (SHMT1), 5-methyltetrahydrofolate-homocysteine methyltransferase reductase (MTRR), and catechol-O-methyltransferase (COMT) can also alter the prevalence of ASD.

Evidence for the association between environmentally-rooted FOCM/TS dysfunction and ASD predisposition can be seen in prenatal valproate and toxic chemical exposure as well as lack of maternal folate supplementation. Maternal valproate use during pregnancy has been associated with higher incidence rates of ASD and in utero valproate exposure has been used to develop rodent models of autism. Valproate exposure can cause DNA hypo-methylation in key neurodevelopmental processes that have been mitigated by folate supplementation in vitro. Other chemicals such as heavy metals, ethyl alcohol, pesticides, phthalates, polychlorinated biphenyls, and traffic-related air pollution (TRAP) can affect neurodevelopment and increase ASD liability. These organic toxins induce oxidative stress and heavy metals disrupt transsulfuration by binding glutathione, the major contributor to intracellular redox homeostasis. Additionally, glutathione is an important regulator in the intracellular processing of methylcobalamin (methyl B₁₂), a cofactor for methionine synthase and the TS pathway. Air dispersion models coupled with traffic patterns/roadway geometry, meteorological data, and vehicle emission data have been used to find a dose response between ASD prevalence and TRAP exposure. Additionally, common organic pollutants have been associated with increased autism severity in children on the autism spectrum.

Latent variable techniques enable the discovery of multivariate interactions, leading to improved classification and regression performance. Furthermore, latent variable techniques allow assessing the importance of individual variables and are more robust to uninformative variables. One example latent variable technique for the classification problems is Fisher Discriminant Analysis (FDA), which can achieve a linear separability using a typically small set of latent variables that are linear combinations of the original variable set. Extensions of FDA, such as Kernel FDA (KFDA), can take nonlinear relationships into account for classification. Latent variable regression techniques include partial least squares (PLS) and its nonlinear counterpart kernel PLS (KPLS). Using FDA for classification and KPLS for regression can allow multivariate interactions to surface, which are often hidden when only univariate analysis is considered. To guarantee a statistically independent assessment of the multivariate classification and regression models, the presented study utilizes a cross-validatory approach, where the set of samples used for model identification does not contain samples to evaluate the performance of the identified models.

The presented work makes use of these advanced modeling and statistical analysis tools to examine metabolite data of the FOCM/TS pathway in neurotypical participants (NEU) and those on the autism spectrum (ASD) as well as their siblings (SIB). Using FDA, the system described herein can distinguish the participants on the spectrum from their neurotypical peers and KPLS unveils a strong correlation between metabolite concentrations of these pathways and adaptive behavior as measured by the Vineland Adaptive Behavior Composite.

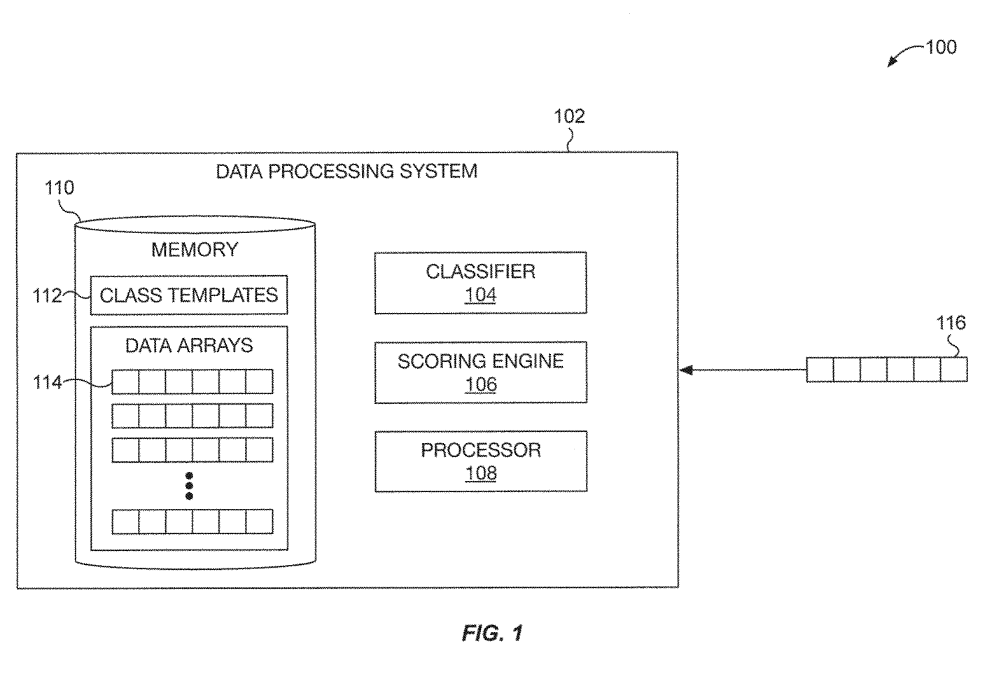

FIG. 1 illustrates a block diagram of an example system 100 to determine an autism state. The system 100 includes a data processing system 102. The data processing system 102 includes a classifier 104 and a scoring engine 106, which are executed by a processor 108. The data processing system 102 also includes a memory 110. Class templates 112 and data arrays 114 are stored on the memory 110. The data processing system 102 is configured to receive a test data array 116.

The data processing system 102 includes the processor 108. The data processing system 102 can include a plurality of processors 108 or other logic devices. The data processing system 102 can be a single entity, such as a laptop or desktop computer or single server. In some implementations, the data processing system 102 can be a distributed system that can include multiple processing systems, such as a cluster of servers that can act in series or parallel to complete the tasks described herein.

The data processing system 102 includes the classifier 104. The classifier 104 can be any script, file, program, application, set of instructions, or computer-executable code, that is configured to enable a computing device on which the classifier 104 is executed to classify incoming data arrays into different autism classes. Example autism classes can include a neurotypical class and an ASD class. The classifier 104 can generate class templates 112 based on training data that includes data arrays 114 from both autistic and healthy patients. The data arrays 114 (and the incoming test data array 116) can each be a vector that includes a plurality of values. Each of the values can represent a different metabolite concentration.

The metabolite concentrations stored in the data arrays 114 can be the concentration of at least one of Methionine, SAM, SAH, SAM/SAH, 8-OHG, Adenosine, Homocysteine, Cysteine, Glu.-Cys., Cys.-Gly., tGSH, fGSH, GSSG, fGSH/GSSG, tGSH/GSSG, Chlorotyrosine, Nitrotyrosine, Tyrosine, Tryptophane, fCystine, fCysteine, fCystine/fCysteine, a percent of DNA methylation, or a percent of oxidized glutathione in a sample obtained from a patient. In some implementations, the data arrays 114 includes a value for each of the above metabolites. In some implementations, the data arrays 114 can include a value for a subpopulation of the metabolites. For example, the data arrays 114 can include values for DNA methylation, 8-OHG, Glu.-Cys., fCystine/fCysteine, Chlorotyrosine, and tGSH/GSSG, and the percent of oxidized glutathione. The order of the concentrations can be arranged in the same order in each of the data arrays 114. For example, the value at a given index n in each of the data arrays 114 can correspond to the same metabolite.

The classifier 104 can generate a class template 112 for the data arrays 114. The classifier 104 can generate the class template 112 using Fisher Discriminant Analysis (FDA). FDA can maximize differences between multiple classes. The classifier 104, using FDA, can determine a linear combination of the values in each of the data arrays 114 that projects the data arrays 114 onto a line joining the mean of the autistic and healthy groups. The classifier 104 calculates the linear combination such that the linear combination projects the data arrays 114 associated with the same class near one another and data arrays 114 associated with the other class disparately. For example, the classifier 104 calculates a linear combination that projects the data arrays 114 into a healthy class and an autistic class. The classifier 104 can save the linear combination as a class template 112. The classifier 104 can also determine a threshold that separates the two classes.

The classifier 104 can use FDA to maximize the difference between the two classes. Specifically, for n samples of m measurements associated with k different classes, the between cluster variability S_{B} is defined to be:

$${S}_{B} = {\sum }_{i = 1}^{k} {n}_{i} ( {\overline{x}}_{i} - \overline{x} ) {( {\overline{x}}_{i} - \overline{x} )}^{T}$$

where x̅ᵢ represents the mean vector of class i, x̅ represents the mean vector of all samples, and n₁ represents the number of samples in class i. The within cluster variation is defined as:

$${S}_{W} = {\sum }_{i = 1}^{k} {n}_{i} {\sum }_{j ∈ i} ( {x}_{i} - {\overline{x}}_{j} ) {( {x}_{j} - {\overline{x}}_{i} )}^{T}$$

wherein xⱼ represents an individual sample FDA. FDA seeks to find at most k−1 vectors that maximize:

$$J ( W ) = \frac{{w}^{T} {S}_{B} w}{{w}^{T} {S}_{w} w}$$

As discussed above, the FDA seeks to find linear combinations of variables that project samples in the same group close to each other and project samples in different groups far away from each other. The solution to this optimization problem is the generalized eigenvectors associated with the k−1 largest generalized eigenvalues of S_{w}⁻¹S_{B}.

The classifier 104 can also calculate a probability distribution function (PDF) of the calculated FDA scores. The classifier 104 can use kernel density estimation to determine the PDF of the FDA scores. The classifier 104 can use the Gaussian kernel:

$$K ( \frac{x - {x}_{i}}{σ} )$$

with each observation xᵢ. Here, x is the additional sample and σ is the kernel parameter that controls the shape of the distribution function. The estimated density function \hat{ƒ}(x) is:

$$\hat{f} ( x ) = \frac{1}{n σ} {\sum }_{i = 1}^{n} K ( \frac{x - {x}_{i}}{σ} )$$

where n is the number of reference samples. The classifier 104 can select the kernel parameter a to minimize the mean integrated squared error (MISE) between the unknown density function ƒ(x) and the estimated density function \hat{ƒ}(x):

MISE(σ)=∫_{-∞}^{∞}(ƒ(x)−\hat{ƒ}(x))²

using a cross-validatory approach.

In some implementations, the classifier 104 can use nonlinear techniques to classify the data arrays into the ASD class and the neurotypical class. For example, the classifier 104 can use kernel partial least squares to classify the data arrays. Kernel techniques provide general nonlinear extensions to the popular linear partial least squares (PLS) regression. The KPLS algorithm commences by defining a nonlinear transformation ƒ=ψ(x) on the predictor set x. In some implementations, ψ(x) can be a Guassian kernel. In some implementations, rather than regress x as a linear PLS, y can be regressed onto the higher dimensional feature space ƒ.

The data processing system 102 includes the scoring engine 106. The scoring engine 106 can be any script, file, program, application, set of instructions, or computer-executable code, that is configured to enable a computing device on which the scoring engine 106 is executed to convert a data array into a score, which is used as a biomarker to categorize the data array 114 into a neurotypical call or ASD class. Upon receiving an test data array 116, the scoring engine 106 can retrieve the class template 112 from the memory 110 and calculate a score for the test data array 116 based on the linear combination stored in the class template 112. The scoring engine 106 can compare the calculated score to the threshold to determine if the test data array 116 should be associated with the neurotypical class or the ASD class.

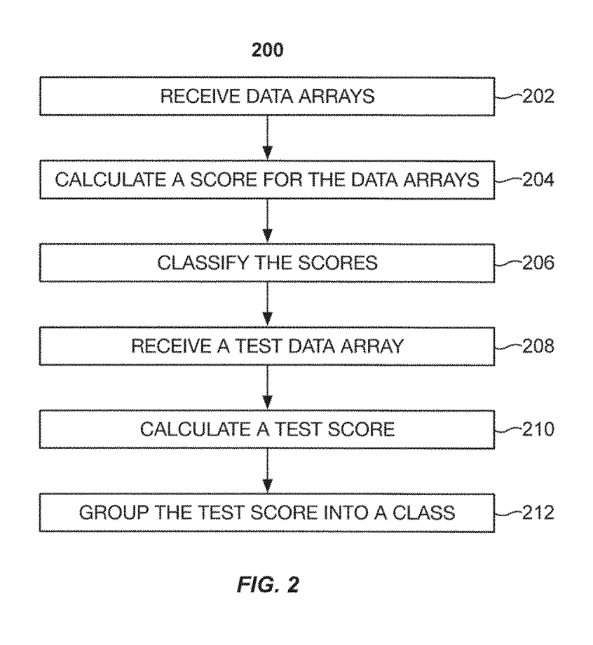

FIG. 2 illustrates a block diagram of an example method 200 for diagnosing autism. The method 200 includes receiving data arrays (ACT 202). The method 200 includes calculating a score for each of the data arrays (ACT 204). The method 200 includes classifying the scores (ACT 206). The method 200 includes receiving a test data array (ACT 208). The method 200 includes calculating a test score (ACT 210). The method 200 also includes grouping the test score into a class (ACT 212).

The method 200 can include receiving data arrays (ACT 202). The data arrays can be vectors that include a plurality of values. The values can each represent a concentration of a different metabolite. Each of the data arrays can be associated with a training subject. A first portion of the data arrays can be identified as belonging to a neurotypical class and a second portion of the data arrays can be identified as belonging to an ASD class. For example, a first bit or value of the data arrays can be set to indicate if the data array belongs to the ASD class.

The metabolites can be Methionine, SAM, SAH, SAM/SAH, 8-OHG, Adenosine, Homocysteine, Cysteine, γ-L-Glutamyl-L-cysteine (Glu.-Cys.), L-Cysteine-L-Glycine (Cys.-Gly.), tGSH, fGSH, GSSG, fGSH/GSSG, tGSH/GSSG, Chlorotyrosine, Nitrotyrosine, Tyrosine, Tryptophane, fCystine, fCysteine, fCystine/fCysteine, a percent of DNA methylation, or a percent of oxidized glutathione. In some implementations, the data arrays consist of the concentration values of DNA methylation, 8-OHG, γ-L-Glutamyl-L-cysteine (Glu.-Cys.), fCystine/fCysteine, Chlorotyrosine, and tGSH/GSSG, and the percent of oxidized glutathione.

The method 200 can include calculating a score for each of the data arrays (ACT 204). The score can be based on relationship between the plurality of values of each of the respective data arrays. For example, to calculate the score, the classifier 104 can perform FDA to generate a linear combination that can project the data arrays identified as belonging to the ASD class into a first group and the data arrays identified as belonging to the neurotypical class into a second group. The linear combination can assign a weight to each of the concentration values in the data arrays.

The method 200 can include classifying the scores of each of the data arrays (ACT 206). The classifier 104 classify the scores into an ASD class or a neurotypical class. In some implementations, the classifier 104 can calculate a PDF of the scores in each of the classes. The classifier 104 can determine a threshold between the PDF for the ASD class and the neurotypical class that separates (or otherwise divides a majority of) the ASD class's PDF from the neurotypical class's PDF. The classifier 104 can save the linear calculation generated during the ACT 204 and the threshold calculated during the ACT 206 into the memory 110.

The method 200 can include receiving a test data array (ACT 208). The test data array can also be referred to as an input data array. The test data array can include a plurality of values that represent the concentration of different metabolites. When a patient is suspected of having autism, a blood test can be performed on the patient to measure the metabolite concentrations in the patient's blood. The test data array can include concentration values for the same metabolites as the data arrays used to train the classifier 104 in ACTS 202-206.

The method 200 can include calculating a test score (ACT 210). The scoring engine 106 can calculate the test score based on a relationship between the test values in the test data array. For example, the scoring engine 106 can retrieve the linear combination stored by the classifier 104 in the memory 110. The scoring engine 106 can use the linear combination to combine the test values and generate a test score. For example, the scoring engine 106 can apply the respective weight of the linear combination to each of the values in the test data array and then combine the weighted values.

The method 200 can include grouping the test score into a class (ACT 212). The test score can be grouped into the ASD class or the neurotypical class. For example, the classifier 104 can retrieve the threshold that separates the ASD class from the neurotypical class. The classifier 104 can compare the test score to the threshold and determine whether the test score is on the ASD class or neurotypical class side of the threshold.

### EXAMPLES

The data used in this example comes from the Arkansas Children's Hospital Research Institute's autism IMAGE study. The protocol was approved by the Institutional Review Board at the University of Arkansas for Medical Sciences and all parents signed informed consent. Subjects between the ages of 3 and 10 years were enrolled to assess levels of oxidative stress. ASD was defined by the Diagnostic and Statistical Manual for Mental Disorders, Fourth Edition, the Autism Diagnostic Observation Schedule (ADOS), and/or the Childhood Autism Rating Scales (CARS; score>30). FOCM/TS metabolites from 83 cases (ASD), 47 siblings (SIB), and 76 age-matched, neurotypical controls (NEU) were used in this example. The metabolites under investigation are tabulated in Table 1. Of the 83 participants on the autism spectrum, 55 also had Vineland II Scores recorded for use in regression analysis (range 46-106). The Vineland Adaptive Behavior Composite evaluates adaptive skills across the domains of communication, socialization, daily living skills, and motor skills through a semi-structured caregiver interview.

Metabolite concentrations were obtained via blood samples taken from each of the subjects. Fasting blood samples were collected before 9:00 am into EDTA-Vacutainer tubes and immediately chilled on ice before centrifuging at 1,300×g for 10 min at 4° C. Aliquots of plasma were transferred into cryostat tubes and stored at −80° C. until extraction and HPLC quantification. The storage interval at −80° C. before extraction was consistently between 1 and 2 weeks after blood draw to minimize potential metabolite inter-conversion. Between-run variation was controlled by inclusion of internal standards with each run. Plasma total folate and vitamin B12 were measured using SimulTRAC-SNB Radioassay Kit for Vitamin B12/Folate from MP Biomedical, Inc. (Orangeburg, N.Y.). The DNA was extracted from whole blood using the Puregene DNA Purification kit (Qiagen, Valencia, Calif.). To ˜1 μg DNA, RNase A (Sigma, St. Louis, Mo.) was added to a final concentration of 0.02 mg/mL and incubated at 37° C. for 15 min. The purified DNA was digested into component nucleotides using nuclease P₁, snake venom phos-phodieasterase, and alkaline phosphatase. DNA base separation and quantification of 5-methylcytosine and cytosine was performed with a Dionex HPLC-UV system coupled to an electrospray ionization (ESI) tandem mass spectrometer (Thermo-Finnigan LCQ) using a Phenomenex Gemini column (C18, 150×2.0 mm, 3 μm particle size) and expressed as percent 5-methylcytosine/total cytosine. The concentration of 8-oxo-deoxyguanosine in DNA was quantified with HPLC electrochemical detection and expressed as pmol/μg DNA.

TABLE 1
FOCM/TS metabolites used in this example

| | | |
|---|---|---|
| Methionine | SAM | SAH |
| SAM/SAH | % DNA methylation | 8-OHG |
| Adenosine | Homocysteine | Cysteine |
| Glu.-Cys. | Cys.-Gly. | tGSH |
| fGSH | GSSG | fGSH/GSSG |
| tGSH/GSSG | Chlorotyrosine | Nitrotyrosine |
| Tyrosine | Tryptophane | fCystine |
| fCysteine | fCystine/fCysteine | % oxidized glutathione |

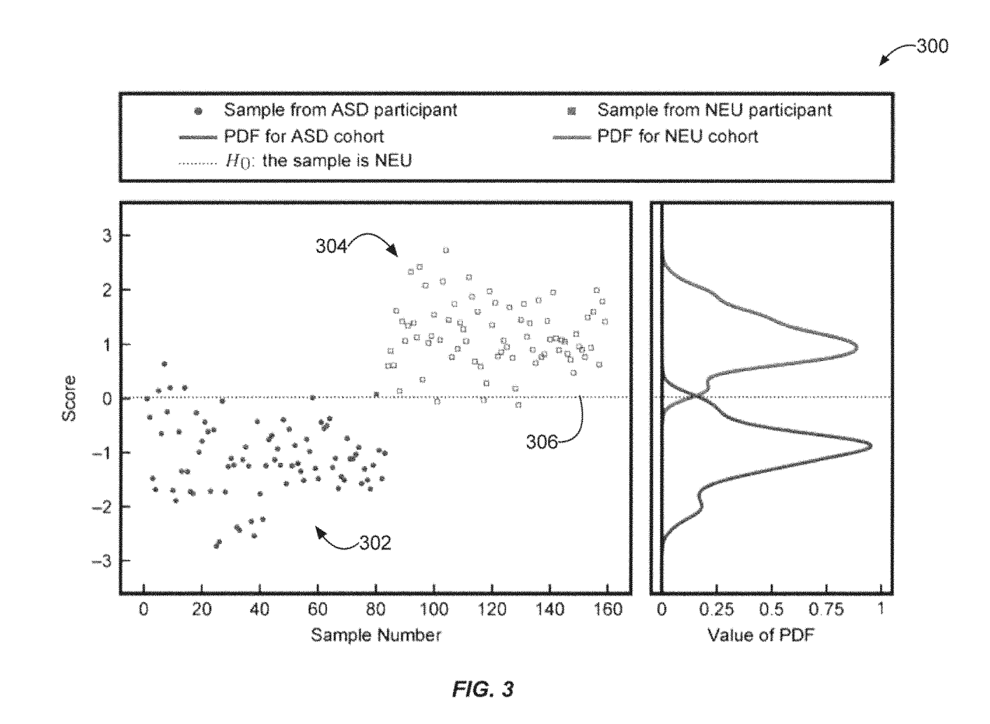

As described above, FDA scores were calculated for each of the patients' data arrays that included the values of the metabolites listed in Table 1. FIG. 3 illustrates a plot 300 of the FDA scores for each of the patients' data arrays and the estimated PDF for each of the classes. The ASD class scores 302 are illustrated as circles and the neurotypical class scores are illustrated as squares 304. The threshold 306 between the two groups is also plotted.

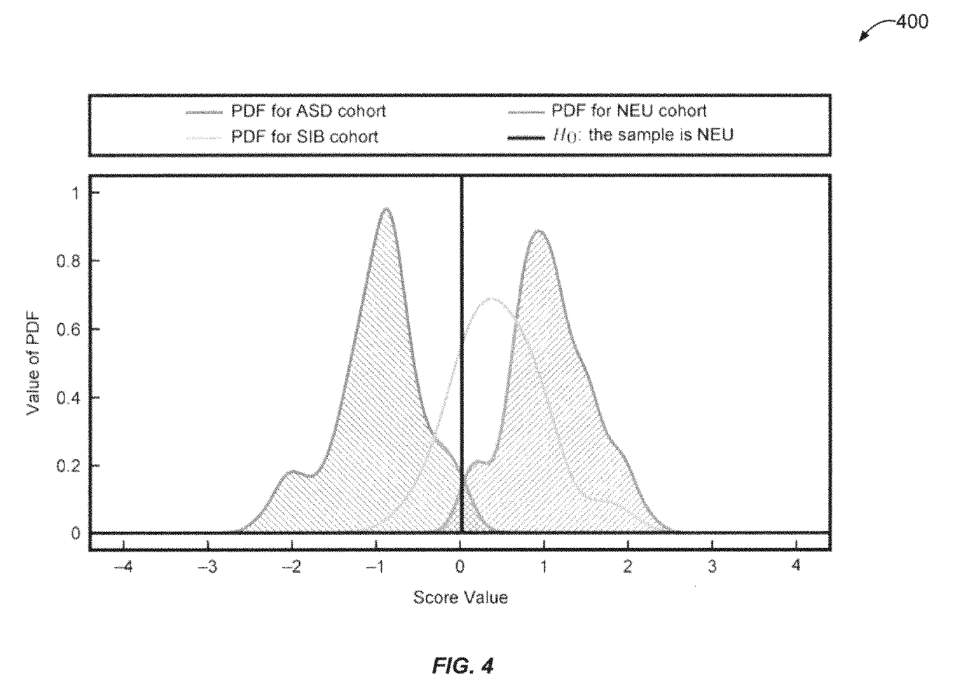

The cross-validated misclassification rates of only 4.9% and 3.4% for the NEU and ASD samples. The performance of the classifier was then evaluated on the SIB class. Evaluation on the SIB class can be a more challenging classification problem due to partially shared genetic and environmental effects with the ASD class. Using all measurements in Table 1, an FDA model was trained to separate the ASD and NEU classes. Then, the trained FDA model was used to evaluate the SIB class (which was not used for training). The resulting separation of ASD, NEU, and SIB presented in FIG. 4. FIG. 4 illustrates a plot 400 of the PDF distributions of the ASD class, the NEU class, and the SIB class. The plot shows a slight increase in the overlap with the ASD class when compared with the performance of the ASD vs. NEU classification.

The simultaneous use of multiple measurements can increase the separability of the classes. However, increasing the number of measurements (e.g., the number of values in the data arrays) can increase the number of parameters in the projection vector w that maximizes the separability of the two groups. Although cross-validation can help mitigate these effects, the increased number of parameters can lead to over-fitting. Over-fitting could indicate good performance for separation on the existing data set, but poor separation performance when the analysis results are translated to new test data. These over-fitting problems can be further mitigated by selecting only the minimum number of variables to adequately separate the two groups. Therefore, all combinations of up to six metabolite concentration values were evaluated for separability. Select combinations of higher numbers of variables were chosen in a greedy fashion to sequentially add measurements that best improve the separation of the best six variables. Cross-validatory FDA was performed on all variable combinations and PDFs of the FDA scores of the two classes were estimated. A receiver-operating-characteristic (ROC) curve was generated based on the PDFs. The C-statistic of the ROC curve can provide a measure of the ability of the classifier to separate into ASD and neurotypical classes. A ROC C-statistic of 0.5 represents random classification and a ROC C-statistic of 1.0 represents perfect classification.

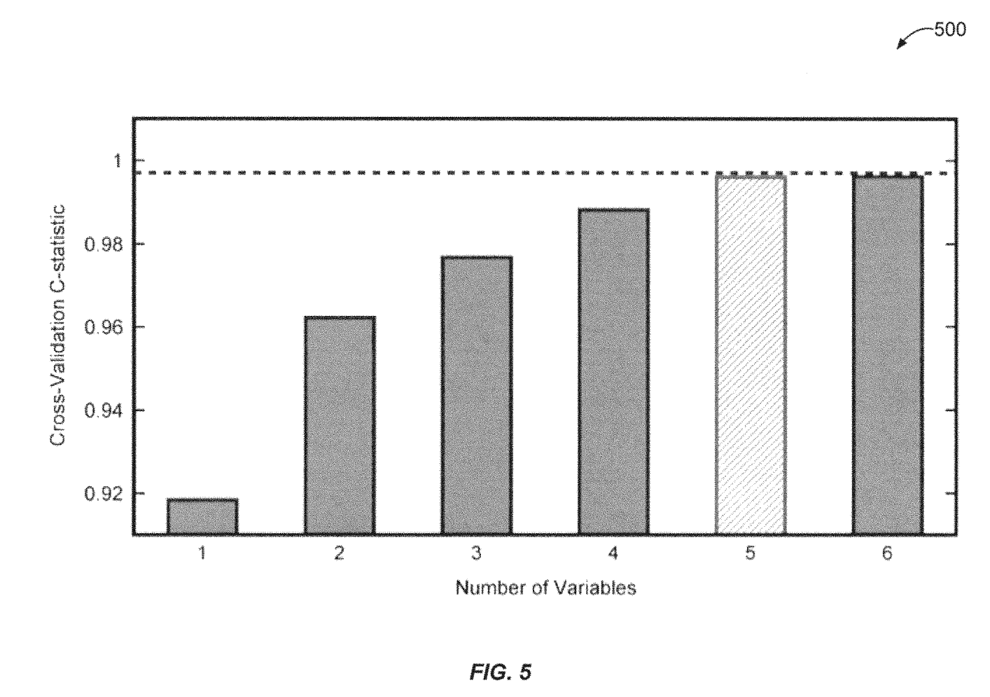

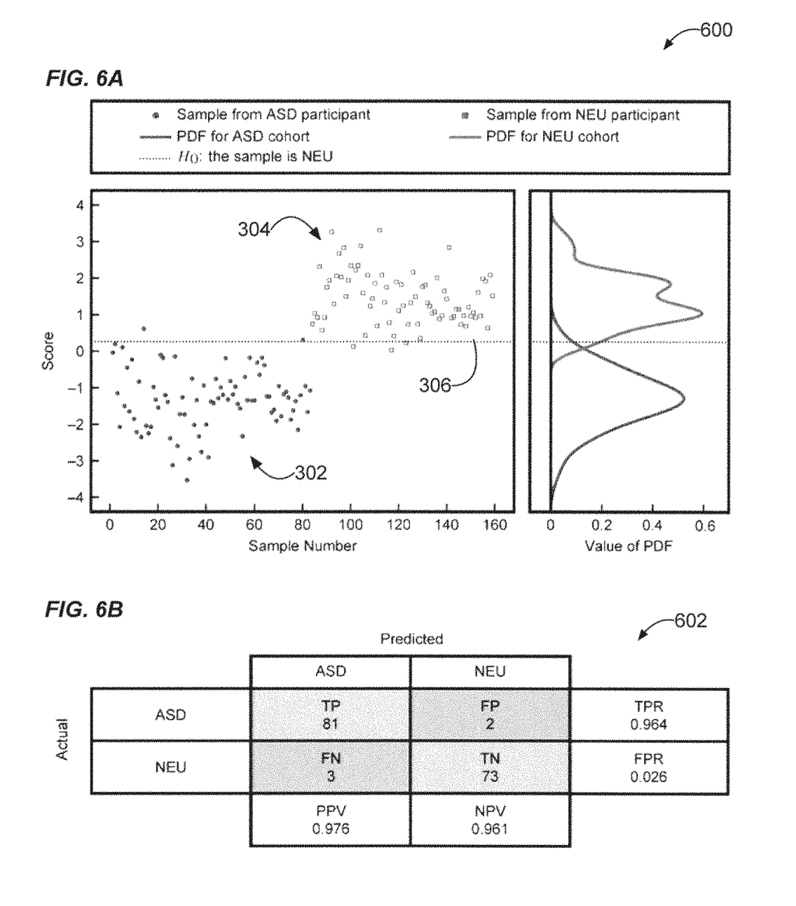

FIG. 5 illustrates a bar plot 500 of the maximum C-statistic for all combinations of a given number of concentration values. As the number of variables increases, the C-statistic increases, saturates at 0.997 for 5 values, and then slightly decreases when over-fitting occurs. From these results, five variables (DNA methylation, 8-OHG, Glu.-Cys., fCystine/fCysteine, % oxidized glutathione) were considered for further analysis. Chlorotyrosine and tGSH/GSSG were added to this set to improve separability of the ASD and SIB groups, increasing the number of metabolites under consideration to seven. The separability of the final minimal classifier based on these seven variables is presented in FIG. 6A.

FIG. 6A illustrates a plot 600 of the FDA scores for each of the patients' data arrays and the estimated PDF for each of the classes using the data arrays with the above reduced number of concentration values. The ASD class scores 302 are illustrated as circles and the neurotypical class scores are illustrated as squares 304. The threshold 306 between the two groups is also plotted. FIG. 6B illustrates the cross-validated confusion matrix 602 for the separation of the ASD and NEU classes. TPR=TP/(TP+FN) is the True Positive Rate, FPR=FP/(FP+TN) is the False Positive Rate, PPV=TP/(TP+FP) is the Positive Predictive Value, and NPV=TN/(TN+FN) is the Negative Predictive Value.

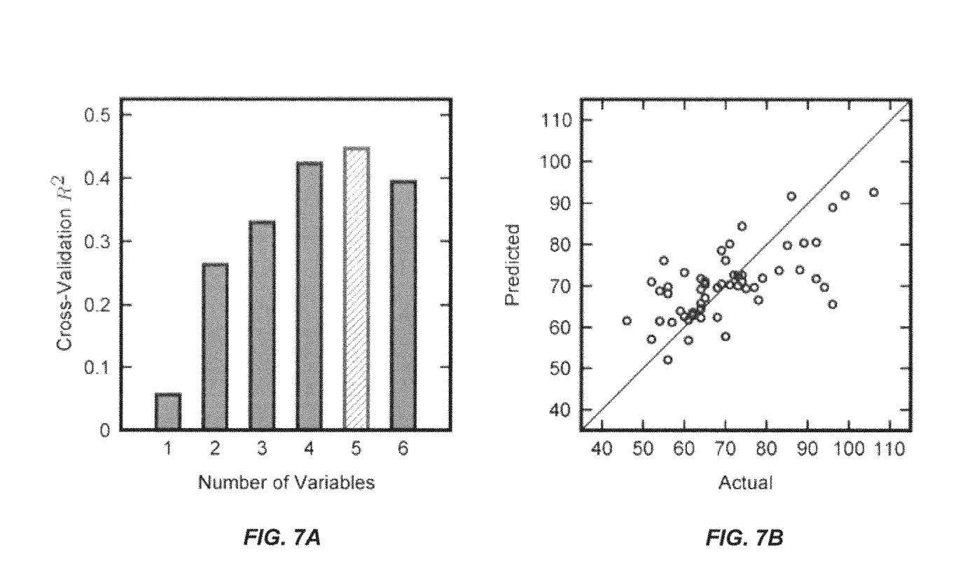

In addition to separation into neurologically distinct classes, the metabolites in the FOCM/TS pathway were investigated for predictability of adaptive behavior. Due to the inter-dependency of pathway metabolites and possible nonlinear effects on psychological outcomes, nonlinear regression via KPLS was used to evaluate the ability of pathway metabolites to predict adaptive behavior in ASD (as measured by the Vineland Adaptive Behavior Composite score). Just as was done in the FDA analysis, all combinations of a given number of variables were evaluated for predictability. The cross-validatory R² of the regression was then used to determine a number of variables in the regression analysis. FIG. 7A illustrates a bar graph of the maximum cross-validated R² for a given number of variables. FIG. 7B illustrates a scatter pot of the cross-validated model predictions versus actual data points for the combination of five variables (GSSG, tGSH/GSSG, Nitrotyrosine, Tyrosine, and fCysteine). From the results illustrated in FIGS. 7A and 7B, the R² begins to decrease when more than five variables are used in the KPLS analysis. The maximum cross-validatory R² was 0.45, corresponding to the KPLS model with the variable combination GSSG, tGSH/GSSG, Nitrotyrosine, Tyrosine, and fCysteine used as inputs. These regression results are plotted in FIGS. 7A and 7B. This strong correlation even after cross-validation indicates the importance of FOCM/TS dysfunction in the pathophysiology of ASD.

The above-described embodiments can be implemented in any of numerous ways. For example, the embodiments can be implemented using hardware, software or a combination thereof. When implemented in software, the software code can be executed on any suitable processor or collection of processors, whether provided in a single computer or distributed among multiple computers.

Also, a computer can have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer can receive input information through speech recognition or in other audible format.

Such computers can be interconnected by one or more networks in any suitable form, including a local area network or a wide area network, such as an enterprise network, an intelligent network (IN) or the Internet. Such networks can be based on any suitable technology and can operate according to any suitable protocol and can include wireless networks, wired networks or fiber optic networks.

A computer employed to implement at least a portion of the functionality described herein can comprise a memory, one or more processing units (also referred to herein simply as “processors”), one or more communication interfaces, one or more display units, and one or more user input devices. The memory can comprise any computer-readable media, and can store computer instructions (also referred to herein as “processor-executable instructions”) for implementing the various functionalities described herein. The processing unit(s) can be used to execute the instructions. The communication interface(s) can be coupled to a wired or wireless network, bus, or other communication means and can therefore allow the computer to transmit communications to and/or receive communications from other devices. The display unit(s) can be provided, for example, to allow a user to view various information in connection with execution of the instructions. The user input device(s) can be provided, for example, to allow the user to make manual adjustments, make selections, enter data or various other information, and/or interact in any of a variety of manners with the processor during execution of the instructions.

The various methods or processes outlined herein can be coded as software that is executable on one or more processors that employ any one of a variety of operating systems or platforms. Additionally, such software can be written using any of a number of suitable programming languages and/or programming or scripting tools, and also can be compiled as executable machine language code or intermediate code that is executed on a framework or virtual machine.

In this respect, various inventive concepts can be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but can be distributed in a modular fashion amongst a number of different computers or processors to implement various aspects of the present invention.

Computer-executable instructions can be in many forms, such as program modules, executed by one or more computers or other devices. Generally, program modules include routines, programs, objects, components, data structures, etc. that perform particular tasks or implement particular abstract data types. Typically the functionality of the program modules can be combined or distributed as desired in various embodiments.

Also, data structures can be stored in computer-readable media in any suitable form. For simplicity of illustration, data structures can be shown to have fields that are related through location in the data structure. Such relationships can likewise be achieved by assigning storage for the fields with locations in a computer-readable medium that conveys relationship between the fields. However, any suitable mechanism can be used to establish a relationship between information in fields of a data structure, including through the use of pointers, tags or other mechanisms that establish relationship between data elements.

Also, various inventive concepts can be embodied as one or more methods, of which an example has been provided. The acts performed as part of the method can be ordered in any suitable way. Accordingly, embodiments can be constructed in which acts are performed in an order different than illustrated, which can include performing some acts simultaneously, even though shown as sequential acts in illustrative embodiments.

As used herein, the term “about” and “substantially” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one” in reference to a list of one or more elements should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03

It will be apparent to those skilled in the art that various modifications and variations can be made in the methods of the present invention without departing from the spirit or scope of the invention. Thus, it is intended that the present invention cover the modifications and variations of this invention provided they come within the scope of the appended claims and their equivalents. All publicly available documents referenced herein, including but not limited to U.S. patents, are specifically incorporated by reference.

What is claimed is:

1. A system to determine an autism state comprising a non-transitory computer storage media, encoded with one or more computer programs, and a processor, the one or more computer programs including a classifier executed by the processor, the classifier configured to:
receive a plurality of data arrays each comprising a plurality of values, wherein each of the plurality of values represent a concentration of a different metabolite;
calculate a score for each of the plurality of data arrays based on a relationship between the plurality of values of each of the respective plurality of data arrays;
classify the score for each of the plurality of data arrays into an autism class and a neurotypical class;
receive a test data array comprising a plurality of test values, wherein each of the plurality of test values represents the concentration of the different metabolites;
calculate a test score for the test data array based on a relationship between the plurality of test values; and
group the test score into one of the autism class and the neurotypical class based on the test score for the test data array.

2. The system of claim 1, wherein each of the plurality of values represent the concentration of Methionine, SAM, SAH, SAM/SAH, 8-OHG, Adenosine, Homocysteine, Cysteine, γ-L-Glutamyl-L-cysteine (Glu.-Cys.), L-Cysteine-L-Glycine (Cys.-Gly.), tGSH, fGSH, GSSG, fGSH/GSSG, tGSH/GSSG, Chlorotyrosine, Nitrotyrosine, Tyrosine, Tryptophane, fCystine, fCysteine, fCystine/fCysteine, a percent of DNA methylation, or a percent of oxidized glutathione, or combinations thereof.

3. The system of claim 1, wherein the plurality of values represent the concentration of each of DNA methylation, 8-OHG, γ-L-Glutamyl-L-cysteine (Glu.-Cys.), fCystine/fCysteine, Chlorotyrosine, and tGSH/GSSG, and the percent of oxidized glutathione.

4. The system of claim 1, wherein the classifier is further configured to:
calculate the score for each of the plurality of data arrays using Fisher Discriminant Analysis, support vector machines, PCA, regression trees, or combinations thereof.

5. The system of claim 1, wherein the classifier is further configured to:
define a border threshold between the autism class and the neurotypical class; and
group, responsive to the test score being below the boarder threshold, the test score into the autism class.

6. The system of claim 5, wherein the boarder threshold is nonlinear.

7. The system of claim 1, wherein the classifier is further configured to:
determine a weight for each of the plurality of values; and
calculate the score for each of the plurality of data arrays using the weight for each of the plurality of values.

8. A computer implemented method to determine an autism state comprising:
receiving a plurality of data arrays each comprising a plurality of values, wherein each of the plurality of values represent a concentration of a different metabolite;
calculating a score for each of the plurality of data arrays based on a relationship between the plurality of values of each of the respective plurality of data arrays;
classifying the score for each of the plurality of data arrays into an autism class and a neurotypical class;
receiving a test data array comprising a plurality of test values, wherein each of the plurality of test values represents the concentration of the different metabolites;
calculating a test score for the test data array based on a relationship between the plurality of test values; and
grouping the test score into one of the autism class and the neurotypical class based on the test score for the test data array.

9. The method of claim 8, wherein each of the plurality of values represent the concentration of Methionine, SAM, SAH, SAM/SAH, 8-OHG, Adenosine, Homocysteine, Cysteine, γ-L-Glutamyl-L-cysteine (Glu.-Cys.), L-Cysteine-L-Glycine (Cys.-Gly.), tGSH, fGSH, GSSG, fGSH/GSSG, tGSH/GSSG, Chlorotyrosine, Nitrotyrosine, Tyrosine, Tryptophane, fCystine, fCysteine, fCystine/fCysteine, a percent of DNA methylation, or a percent of oxidized glutathione, or combinations thereof.

10. The method of claim 8, wherein the plurality of values represent the concentration of each of DNA methylation, 8-OHG, γ-L-Glutamyl-L-cysteine (Glu.-Cys.), fCystine/fCysteine, Chlorotyrosine, and tGSH/GSSG, and the percent of oxidized glutathione.

11. The method of claim 8, further comprising:
calculating the score for each of the plurality of data arrays using Fisher Discriminant Analysis, support vector machines, PCA, regression trees, or combinations thereof.

12. The method of claim 8, further comprising:
defining a boarder threshold between the autism class and the neurotypical class; and
grouping, responsive to the test score being below the boarder threshold, the test score into the autism class.

13. The method of claim 12, wherein the boarder threshold is nonlinear.

14. The method of claim 8, further comprising:
determining a weight for each of the plurality of values; and
calculating the score for each of the plurality of data arrays using the weight for each of the plurality of values.
